(19) United States
(12) Patent Application Publication
(10) Pub. No.: US 2009/0204049 A1
(43) Pub. Date: Aug. 13, 2009

(54) BLOOD CONTAINER AND METHOD FOR A BLOOD TRANSFUSION

(76) Inventor: Song Lee, Seoul (KR)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/368,745

(22) Filed: Feb. 10, 2009

(30) Foreign Application Priority Data
Feb. 12, 2008 (KR) 10-2008-12721

(51) Int. Cl. A61M 1/02; A61J 1/05

(52) U.S. Cl. 604/5.03; 604/403; 604/404

(57) ABSTRACT

A blood container and a method is provided for a blood transfusion, in particular, for a blood transfusion by which blood a patient is losing is collected, fat is separated from the blood, and the non-fat blood is transfused into the patient so as to prevent side-effects including fat embolism and the like from occurring due to the fat after the blood is transfused into the patient. The blood container includes a soft case and hard case. A first tube is formed at one side of the hard case to inject blood into the soft and hard cases and a second tube is formed adjacent to the first tube to discharge the blood out of the soft and hard cases, wherein the soft and hard cases have open side that adhere to each other to form a single body.

### CROSS-REFERENCE TO RELATED APPLICATION

This application claims the benefit of Korean Patent Application No. 10-2008-0012721, filed on Feb. 12, 2008, the disclosure of which is incorporated herein in its entirety by reference.

### BACKGROUND

The present invention relates to a blood container and a method for a blood transfusion, and more particularly, to a blood container and a method for a blood transfusion by which blood a patient is losing is collected, fat is separated from the blood, and the non-fat blood is re-transfused into the patient so as to prevent side-effects, such as fat embolism and the like, which are likely to occur due to the fat after the blood is transfused.

Hospitals or blood centers have lack of blood with the decreases in blood donations. Thus, they do not frequently cope with emergent situations in that blood is to be transfused into patients who have lost a large amount of blood due to traumatic injuries caused by traffic accidents, etc. Therefore, if a patient, who requires an emergent blood transfusion due to a great loss of blood, occurs, a self-blood transfusion is performed with respect to the patient to collect the blood the patient is losing so as to re-transfuse the collected blood into the patient.

If a patient is transfused with his or her blood as described above, the patient can be prevented from dangers, such as viral hepatitis, Acquired Immune Deficiency Syndrome (AIDS), etc., and hemolytic, pyrexial, and allergic transfusion side-effects. In this case, collected blood passes through a predetermined machine so as to remove foreign materials from the blood and then is transfused into the patient. However, fat remains in the blood after the foreign materials are removed from the blood. The fat remaining in the blood conglomerates when it passes through the machine or is transfused into the patient. Thus, the remaining fat forms droplets, which increases the bulk of the fat. Also, the fat droplets stop a capillary vessel of the patient after the blood is transfused into the body of the patient. As a result, side-effects, such as fat embolism and the like, occur in the patient, which may threaten the life of the patient.

In order to solve these problems, an appropriate amount of a saline solution is added into a blood container which contains blood which has passed through a machine, so as to separate fat from the flood using a weight difference between the fat and the saline solution contained in the blood container and then transfuse the non-fat blood into the patient. However, if the blood container is not removed from the patient at an appropriate time, the blood contained in the blood container may be all injected into the patient. As a result, the fat, which has been separated from the blood, may be all injected into the patient.

In addition, there have been developed filters which filter off fat from blood. However, since the filters are too high-priced, and patients are to cover great expense.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with an aspect of one embodiment, a blood container is provided to contain blood and to include a hard case to prevent fat separated from the blood from being injected into a patient in order to prevent side-effects from occurring due to the fat after the blood is transfused into the patient.

In accordance with another aspect of one embodiment, a method is provided for a blood transfusion by which a predetermined amount of a saline solution is added into a blood container including a body of which part is formed of a hard case, fat is separated from blood, the non-fat blood is transfused into a patient, and the transfusion of the blood stops when a predetermined amount of the blood and the separated fat remain in the hard case of the blood container, so as to prevent side-effects, such as fat embolism and the like, from occurring after the blood is transfused into the patient.

In accordance with yet another aspect of one embodiment, there is provided a blood container including: a soft case of which side is opened; a hard case of which side is opened so as to be connected to the soft case; a first tube which is formed at an other side of the hard case so as to inject blood into the soft and hard cases; and a second tube which is formed adjacent to the first tube so as to discharge the blood out of the soft and hard cases, wherein the opened sides of the soft and hard cases adhere to each other so as to form a single body.

In one aspect, there is provided a blood container including: a soft case of which inside is sealed; a hard case into which an end of the soft case is inserted and thus which adheres to the soft case; a first tube which is formed at the end of the soft case so as to inject blood into the soft case; and a second tube which is formed adjacent to the first tube so as to discharge the blood out of the soft case.

In another aspect, a blood transfusion method is provided where blood a patient is losing is collected, and then foreign materials are removed from the collected blood in order to re-transfuse the blood into the body of the patient. The method includes injecting the blood, from which the foreign materials have been removed, into a blood container comprising a hard case formed at an end of a body of the blood container; separating fat from the blood contained in the blood container by adding a saline solution into the blood container and transfusing the blood, from which the fat has been separated, into the patient, wherein if a predetermined amount of blood from which the fat has been separated remains in the hard case in the transfusion of the blood into the patient, the hard case maintains its original shape so as to stop transfusing the blood.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an exploded perspective view of a blood container according to an embodiment of the present invention;

FIG. 2 is a perspective view of a blood container according to another embodiment of the present invention;

FIG. 3 is a perspective view of a blood container according to another embodiment of the present invention; and

FIG. 4 is a perspective view of a blood container according to another embodiment of the present invention.

### DETAILED DESCRIPTION

While illustrative embodiments have been illustrated and described, it will be appreciated that various changes can be made therein without departing from the spirit and scope of the invention.

The present invention will now be described more fully with reference to the accompanying drawings, in which exemplary embodiments of the invention are shown.

The invention may, however, be embodied in many different forms and should not be construed as being limited to the embodiments set forth herein.

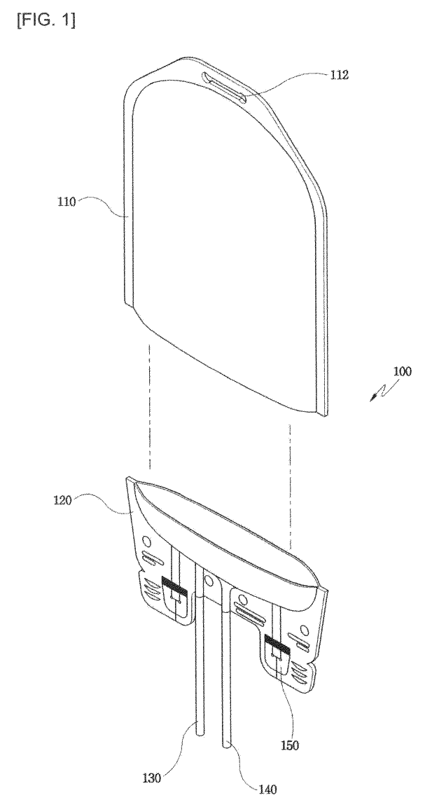

FIG. 1 is an exploded perspective view of a blood container according to an embodiment of the present invention. Referring to FIG. 1, the blood container 100 according to the present embodiment includes a soft case 110, a hard case 120, a first tube 130, a second tube 140, and an inlet 150. The soft case 110 includes a space which is necessary for containing blood and an opening which is formed at a side of the soft case 110. The hard case 120 is connected to the opening of the soft case 110 so as to form a single body with the soft case 110. The first tube 130 is formed at a side of the hard case 120 so as to inject the blood into the soft and hard cases 110 and 120. The second tube 140 is formed adjacent to the first tube 130 so as to discharge the blood out of the soft and hard cases 110 and 120. The inlet 150 is formed adjacent to the first or second tube 130 or 140 so as to inject an additive into the soft and hard cases 110 and 120.

The soft case 110 includes the space which is necessary for containing the blood, the opening which is formed at the side of the soft case 110, and a throughhole 112 which is formed at the other side of the soft case 110 so as to hang the soft case 110 on a stand during a blood transfusion.

The soft case 110 is formed of polyvinyl chloride (PVC), which is generally used as a material of which a blood container is formed, to have softness so as to be deformed by an external force or pressure.

The hard case 120 include a space which is necessary for containing the blood, an opening which is formed at a side of the hard case 120, the first tube 130 is formed at an other side of the hard case 120 so as to inject the blood into the blood container 100, the second tube 140 is formed at the other side of the hard case 120 so as to discharge the blood out of the blood container 100, and the inlet 150 which is formed at the other side of the hard case 120 so as to inject the additive into the blood container 100.

The hard case 120 is formed of a PVC or plastic material having hardness so as not to be deformed by an external force or pressure. The hard case 120 may be formed to a height enough to contain the blood at a height from 1 cm to 3 cm. Since the hard case 120 preserves its original shape from pressure generated during the blood transfusion, blood remaining in the hard case 120, i.e., blood containing fat, is not injected into the body of a patient.

The soft and hard cases 110 and 120 are connected to each other to form a single body. Here, the soft and hard cases 110 and 120 may be fused to each other through a heat treatment or may be connected to each other using an adhesive which is not harmful to a human body.

The first and second tubes 130 and 140 are formed of a material having softness such as silicon, rubber, or the like. Alternatively, a valve may be interposed in each of the first and second tubes 130 and 140 in order to prevent blood from flowing backward.

The inlet 150 is formed so as to inject an additive such as an anticoagulant, a saline solution, or the like. An upper part of the inlet 150 may be formed of a soft material to insert an injector into the inlet 150 so as to inject the additive into the blood container 100.

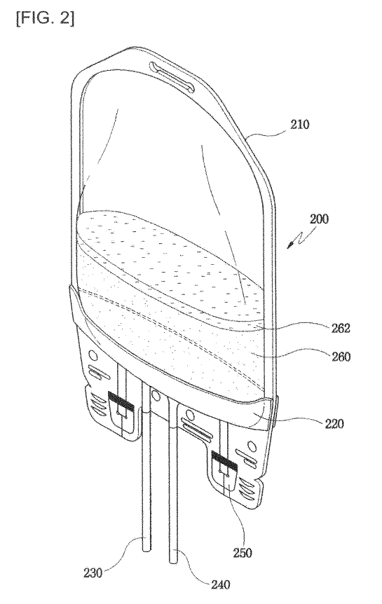

FIG. 2 is a perspective view of a blood container according to another embodiment of the present invention. Referring to FIG. 2, the blood container 200 according to the present embodiment includes a soft case 210, a hard case 220, a first tube 230, a second tube 240, and an inlet 250. The soft case 210 includes an airtight space which is formed to contain blood. A side of the soft case 210 is inserted into the hard case 220. The first tube 230 is formed at the side of the soft case 210 so as to inject the blood into the soft case 210. The second tube 240 is formed adjacent to the first tube 230 so as to discharge the blood out of the soft case 210. The inlet 250 is formed adjacent to the first or second tube 230 or 240 so as to inject an additive into the soft case 210.

The soft case 210, the first tube 230, the second tube 240, and the inlet 250 are formed of the same material as that of which a general blood container is formed and have the same structures as those of a soft case, a first tube, a second tube, and an inlet of the general blood container.

The hard case 220 has a structure so that the side of the soft case 210 is inserted into or adhered to the hard case 220 and is formed of a PVC or plastic material having hardness. The hard case 220 may be adhered to the soft case 210 using an adhesive agent such as an adhesive, an adhesive tape, or the like. Here, the adhesive agent may be coated on the whole part of an inside of the hard case 220 so as to prevent the soft case 210 from being fused to the hard case 220 during a blood transfusion. The hard case 220 may be formed to a height enough to store the blood at a height from 1 cm to 3 cm in the soft case 210.

The first tube 230, the second tube 240, and the inlet 250 may be formed of the same materials as those of which the first tube 130, the second tube 140, and the inlet 150 according to the previous embodiment are formed and may have the same structures as the first tube 130, the second tube 140, and the inlet 150.

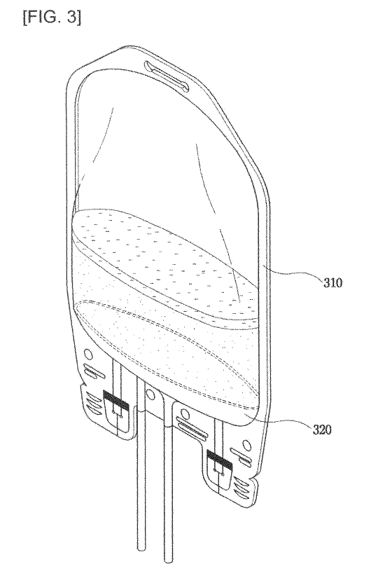

FIG. 3 is a perspective view of a blood container according to another embodiment of the present invention. Referring to FIG. 3, a hard case 320 is inserted into a soft case 310 differently from the blood container 200 of the previous embodiment. When the soft case 310 is manufactured, the hard case 320 is inserted into the soft case 310, and then the soft case 310 is sealed. When the hard case 320 is inserted into the soft case 310, the hard case 320 is positioned underneath the soft case 310 during a blood transfusion due to its weight. Since the hard case 320 maintains its original shape during the blood transfusion, most of blood contained in the soft case 310 is injected into the body of a patient, and blood remaining in the hard case 320, i.e., blood containing fat separated from the blood, is not injected into the body of the patient.

The hard case 320 is formed of a PVC or plastic material. The hard case 320 may be formed to a height enough to store the blood at a height from 1 cm to 3 cm in the soft case 310.

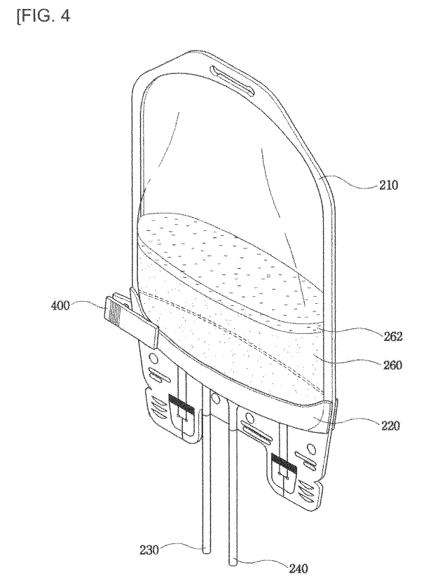

FIG. 4 is a perspective view of a blood container according to another embodiment of the present invention. Referring to FIG. 4, the blood container of the present embodiment has the same structure as the blood container 100, 200, or 300 according to the previous embodiment. The blood container of the present embodiment further includes a sensor 400 which measures an amount of blood remaining in the blood container and informs a medical staff of the remaining amount of the blood.

When the blood reaches a predetermined position inside a hard case 220 during a blood transfusion, the sensor 400 measures a position of the blood and informs the medical staff of the position of the blood using a warning sound or the like.

The sensor 400 installed at the hard case 220 may be an optical sensor. If the optical sensor is installed at the hard case 220, throughholes are formed at a predetermined position of the hard case 220. In other words, the throughholes are formed at both sides of the hard case 220 so as to face each other, so that a light emitting sensor emitting light and a light receiving sensor receiving the light are opposite to each other through the throughholes.

If the blood reaches the predetermined position of the hard case 220 during the blood transfusion, the light receiving sensor receives the light from the light emitting sensor. In this case, the sensor 400 generates the warning sound through an additional acoustic device connected to the light receiving sensor in order to inform the medical staff that the light receiving sensor has received the light. Thus, the medical staff recognizes a replacement or removal time of the blood container through the warning sound. As a result, fat separated from the blood is prevented from flowing into the body of a patient.

A method for a blood transfusion according to an embodiment of the present invention will now be described with reference to FIG. 2.

Blood 260 a patient is losing is collected and then passes through a predetermined machine so as to remove foreign materials. Thereafter, the blood 260 is injected into the blood container 200 having the above-described structure.

A predetermined amount of a saline solution is injected into the blood container 200.

When the blood container 200 is reversed, fat is separated from the blood 260 so as to form a fat layer 262 on the blood 260. Here, since densities of the blood 260, the saline solution, and the fat contained in the blood container 200 are different from one another, the fat having the lowest density floats on the blood 260 and the saline solution. Thus, the fat separated from the blood 260 forms the fat layer 262 which floats in the uppermost position of the blood container 200.

When the blood container 200 is connected to the patient through the second tube 240, the blood 260 is injected into a blood vessel of the patient due to a pressure difference. In other words, the blood 260 is injected into the blood vessel of the patient with the soft case 210 of the blood container 200 compressed. Here, the blood 260 injected into the blood vessel of the patient contains a predetermined amount of the saline solution.

When a predetermined amount of the blood 260 is injected into the patient and then reaches the hard case 220, the blood container 200 is not compressed any more. Thus, the transfusion of the blood 260 is stopped, and thus a residual amount of the blood 260 in the hard case 220 is not injected into the patient. As a result, the fat layer 262 in the blood container 200 is prevented from flowing into the patient. Here, if the sensor 400 is installed at the blood container 200, the sensor 400 generates a warning sound so as to inform a medical staff that the transfusion of the blood 260 into the patient has stopped at the hard case 220.

In a blood container and a method for a blood transfusion according to the present invention, a hard case is not fused during a blood transfusion. Thus, fat separated from the blood is prevented from being supplied into the body of a patient. As a result, side-effects, such as fat embolism and the like, are prevented from occurring due to the fat after the blood is transfused into the body of the patient.

While the present invention has been particularly shown and described with reference to exemplary embodiments thereof, it will be understood by those of ordinary skill in the art that various changes in form and details may be made therein without departing from the spirit and scope of the present invention as defined by the following claims.

The embodiments of the invention in which an exclusive property or privilege is claimed are defined as follows:

1. A blood container comprising:
a soft case of which side is opened;
a hard case of which side is opened so as to be connected to the soft case;
a first tube which is formed at an other side of the hard case so as to inject blood into the soft and hard cases; and
a second tube which is formed adjacent to the first tube so as to discharge the blood out of the soft and hard cases,
wherein the opened sides of the soft and hard cases adhere to each other so as to form a single body.

2. The blood container of claim 1, further comprising an inlet through which an additive is injected.

3. The blood container of claim 1, wherein the hard case is formed of a plastic material.

4. The blood container of claim 1, wherein the hard case is formed to a height enough to store blood at a height from 1 cm to 3 cm in the soft case.

5. The blood container of claim 1, wherein the hard case is connected to an end of the soft case through a heat treatment.

6. A blood container comprising:
a soft case of which inside is sealed;
a hard case into which an end of the soft case is inserted and thus which adheres to the soft case;
a first tube which is formed at the end of the soft case so as to inject blood into the soft case; and
a second tube which is formed adjacent to the first tube so as to discharge the blood out of the soft case.

7. The blood container of claim 6, further comprising an inlet through which an additive is injected.

8. The blood container of claim 6, wherein the hard case is formed of a plastic material.

9. The blood container of claim 6, wherein the hard case is formed to a height enough to store blood at a height from 1 cm to 3 cm in the soft case.

10. The blood container of claim 6, wherein the hard case adheres onto a surface of the soft case using an adhesive.

11. A blood container comprising:
a soft case of which inside is sealed;
a hard case which is inserted into the soft case;
a first tube which is formed at an end of the soft case so as to inject blood into the soft case; and
a second tube which is formed adjacent to the first tube so as to discharge the blood out of the soft case.

12. The blood container of claim 11, further comprising an inlet through which an additive is injected.

13. The blood container of claim 11, wherein the hard case is formed of a plastic material.

14. The blood container of claim 11, wherein the hard case is formed to a height enough to store blood at a height from 1 cm to 3 cm in the soft case.

15. The blood container of claim 11, wherein the hard case comprises a sensor which measures an amount of blood remaining in the hard case.

16. A method for blood transfusion by which blood a patient is losing is collected, and then foreign materials are removed from the collected blood in order to re-transfuse the blood into the body of the patient, comprising:
injecting the blood, from which the foreign materials have been removed, into a blood container comprising a hard case formed at an end of a body of the blood container;
separating fat from the blood contained in the blood container by adding a saline solution into the blood container; and
transfusing the blood, from which the fat has been separated, into the patient,
wherein if a predetermined amount of blood from which the fat has been separated remains in the hard case in the transfusion of the blood into the patient, the hard case maintains its original shape so as to stop transfusing the blood.

17. The method of claim 16, wherein the separation of the fat from the blood is performed by injecting the saline solution into the blood container and then reversing the blood container in order to position the fat above the saline solution due to a difference between densities of the fat and the saline solution contained in the blood container.
